(19) United States
(12) Patent Application Publication
(10) Pub. No.: US 2024/0226034 A1
(43) Pub. Date: Jul. 11, 2024

(54) Use of Terpenoids and Salicylates as Anesthetics and Analgesics

(71) Applicant: THE REGENTS OF THE UNIVERSITY OF CALIFORNIA, OAKLAND, CA (US)

(72) Inventor: Robert Brosnan, Oakland, CA (US)

(73) Assignee: THE REGENTS OF THE UNIVERSITY OF CALIFORNIA, OAKLAND, CA (US)

(21) Appl. No.: 18/557,258

(22) PCT Filed: Apr. 27, 2022
(86) PCT No.: PCT/US2022/026482

§ 371 Date: Oct. 25, 2023

Related U.S. Application Data
(60) Provisional application No. 63/181,927, filed on Apr. 29, 2021.

(51) Int. Cl. A61K 31/122; A61K 9/00; A61K 9/107; A61K 31/603; A61K 47/40; A61P 23/00

(52) U.S. Cl. CPC A61K 31/122; A61K 9/0019; A61K 9/107; A61K 31/603; A61K 47/40; A61P 23/00

(57) ABSTRACT

Provided herein are compositions for inducing anesthesia in a subject, where the compositions include at least one compound that is either a terpenoid or a salicylate. The provided compositions are particularly useful for applications where the subject is a livestock animal used for meat or milk production, as the anesthetic agents have no toxic effects on food byproducts. The compositions provide further advantages for applications where the subject is a human undergoing a medical procedure. Also provided are methods for using the disclosed compositions and compounds.

### CROSS-REFERENCE TO RELATED APPLICATIONS

The present application claims priority to U.S. Provisional Application No. 63/181,927 filed Apr. 29, 2021, the full disclosure of which is incorporated by reference in its entirety for all purposes.

### BACKGROUND

Anesthesia in humans enables the performance of medical operations that would otherwise cause severe or intolerable pain to patients, or that would otherwise be technically infeasible. Anesthesia can block transmission of nerve impulses from specific parts of the body, can inhibit anxiety and the creation of long-term memories, and can result in unconsciousness and a total lack of sensation. Additionally, veterinary anesthesia performed on non-human animals is useful for an even wider range of circumstances, as these animals have less ability to cooperate with certain diagnostic or therapeutic procedures. Further, anesthetic agents find additional use in non-human animals for purpose of euthanasia of veterinary species. The wide-ranging benefits of anesthesia must be weighed, however, against risks or other drawbacks associated with the use of various anesthetic agents. For example, many such agents have recognized addictive attributes, contribute to environmental contamination, and can render food byproducts of anesthetized animals at least temporarily unfit for consumption. Indeed, the use of barbiturates such as pentobarbital as veterinary euthanasia agents has introduced substantial amounts of slow-decomposing contaminants into the food chain and groundwater environment.

Because of their potential for abuse, certain chemical agents have been identified by the United States Department of Justice Drug Enforcement Administration as being controlled substances, and categorized into five schedules according to abusers' physical and psychological dependence on the agents. Controlled substances commonly used in vertebrate animal research and veterinary care include butorphanol, a schedule 4 analgesic; ketamine, a schedule 3 anesthetic agent; buprenorphine, a schedule 3 narcotic analgesic; and sodium pentobarbital, a schedule 2 anesthetic and euthanasia agent. Such controlled substances are heavily regulated, with strict procedural requirements in place for their authorization, purchase, storage, logging, and disposal.

Injectable anesthetic agents enter the environment in general, and drinking water in particular, either directly through the disposal of unused drugs, or indirectly through human excretion. Furthermore, low decomposition rates of these agents can lead to a substantial environmental persistence having largely unknown effects on human, animal, and plant health. Inhalation anesthetic agents pose other environmental risks associated with their atmospheric impacts. Nitrous oxide has been shown to contribute significantly to global warming and ozone depletion, and sevoflurane, isolflurane, and desflurane each have a global warming potential that is two to three orders of magnitude greater than that of carbon dioxide. Estimates place the contribution of exhaled anesthetic agents to the overall carbon footprint of some medical organizations at approximately 5%.

Administration of an anesthetic agent having toxicity effects to a meat or milk producing animal necessitates a withdrawal time period for the agent to be metabolized by the animal and the concentration of the agent within the animal tissues to decrease. Without this withholding time, foods produced from the animal will have levels of the toxic agent too high for safe human consumption, and the meat or milk will need to be disposed. Withdrawal or withholding times for different agents vary with the pharmacokinetics of the particular agent, but can be as long as several days.

In view of these and other challenges associated with the use of many existing anesthetic agents, there is a need in the art for improved chemical compounds and compositions that can be used with anesthesiology methods in humans and non-human animals. The present disclosure addresses this need and provides associated and other advantages.

### BRIEF SUMMARY

In general, provided herein are improved anesthesiology methods and compositions that are highly effective in achieving a desired endpoint while also having reduced environmental and toxicity concerns and lower potentials for substance abuse. In one aspect, the disclosure provides a method of inducing anesthesia in a subject. The method includes administering to the subject an amount of a compound that includes one or both of a terpenoid and a salicylate. The amount administered is effective for inducing anesthesia in the subject.

In some embodiments, the compound of the method is a Type II voltage-gated sodium channel antagonist. In some embodiments, the compound is an N-methyl-D-aspartate receptor antagonist. In some embodiments, the compound is a γ-aminobutyric acid type A (GABA_{A}) receptor agonist. In some embodiments, the compound is a terpenoid. In some embodiments, the terpenoid is a hemiterpenoid. In some embodiments, the terpenoid has two isoprene units. The terpenoid can be, for example, L-carvone. In some embodiments, the terpenoid has three isoprene units. In some embodiments, the compound is a salicylate. In some embodiments, the salicylate is an ester of salicylic acid. The salicylate can be, for example, methyl salicylate, ethyl salicylate, or benzyl salicylate.

In some embodiments, the subject of the method is a mammal. In some embodiments, the mammal is a livestock animal used for meat, milk, or wool production. In some embodiments, the mammal is a dog, cat, rabbit, rodent, horse, or other companion animal. In some embodiments, the mammal is a human.

In some embodiments, the administering of the compound to the subject is via injection. The injection can be, for example, intravenous. In some embodiments, the effective amount administered includes a dosage less than or equal to 2 mg/kg. In some embodiments, the effective amount includes a dosage of from 2 mg/kg to 4 mg/kg. In some embodiments, the effective amount includes a dosage greater than or equal to 4 mg/kg. In some embodiments, the anesthesia of the method has an endpoint including one or more of analgesia, tranquilization, sedation, amnesia, a hypnotic state, and a state of insensitivity to noxious stimulation. In some embodiments, the anesthesia has an endpoint including analgesia. In some embodiments, the anesthesia is general anesthesia. In some embodiments, the anesthesia has an endpoint including euthanasia.

In some embodiments, the compound of the method is formulated in a composition including a delivery vehicle. In some embodiments, the delivery vehicle includes a lipid emulsion, e.g., INTRALIPID®. In some embodiments, the delivery vehicle includes a cyclodextrin.

In another aspect, the disclosure provides a composition for inducing anesthesia in a subject. The composition includes an amount of a compound that includes one or both of a terpenoid and a salicylate. The amount of the compound is effective for inducing anesthesia in the subject. The composition further includes a delivery vehicle.

In some embodiments, the compound of the composition is a Type II voltage-gated sodium channel antagonist. In some embodiments, the compound is an N-methyl-D-aspartate receptor antagonist. In some embodiments, the compound is a GABA_{A} receptor agonist. In some embodiments, the compound is a terpenoid. In some embodiments, the terpenoid is a hemiterpenoid. In some embodiments, the terpenoid has two isoprene units. The terpenoid can be, for example, L-carvone. In some embodiments, the terpenoid has three isoprene units. In some embodiments, the compound is a salicylate. In some embodiments, the salicylate is an ester of salicylic acid. The salicylate can be, for example, methyl salicylate, ethyl salicylate, or benzyl salicylate.

In some embodiments, the subject receiving the composition is a mammal. In some embodiments, the mammal is a livestock animal used for meat, milk, or wool production. In some embodiments, the mammal is a dog, cat, rabbit, rodent, horse, or other companion animal. In some embodiments, the mammal is a human.

In some embodiments, the effective amount of the compound in the composition includes a dosage less than or equal to 2 mg/kg. In some embodiments, the effective amount includes a dosage of from 2 mg/kg to 4 mg/kg. In some embodiments, the effective amount includes a dosage greater than or equal to 4 mg/kg. In some embodiments, the anesthesia of the method has an endpoint including analgesia, tranquilization, sedation, amnesia, a hypnotic state, a state of insensitivity to noxious stimulation, or a combination thereof. In some embodiments, the anesthesia has an endpoint including analgesia. In some embodiments, the anesthesia is general anesthesia. In some embodiments, the anesthesia has an endpoint including euthanasia.

In some embodiments, the delivery vehicle of the composition includes a lipid emulsion. In some embodiments, the delivery vehicle includes a cyclodextrin.

### BRIEF DESCRIPTION OF THE DRAWINGS

FIG. 1 is a sample electrophysiology tracing for GABA_{A} (γ-amino butyric acid type A) receptors before, during, and after exposure to 1 mML-carvone. Tracings for methyl salicylate exposures were qualitatively similar.

FIG. 2 is a sample electrophysiology tracing for NMDA (N-methyl-D-aspartate) receptors before, during, and after exposure to 1 mML-carvone. Tracings for methyl salicylate exposures were qualitatively similar.

FIG. 3 is a sample electrophysiology tracing for Naᵥ1.2 (type-2 voltage-gated sodium) channels before, during, and after exposure to 1 mML-carvone. Tracings for methyl salicylate exposures were qualitatively similar.

FIG. 4 is a table of bootstrap parameter estimates for the nonlinear regression fit of the percent current change to the Hill equation:

$${I}_{max} \frac{{C}^{{n}_{I}}}{{IC}_{50}^{{n}_{I}} + {C}^{{n}_{I}}} + {E}_{max} \frac{{C}^{{n}_{E}}}{{IC}_{50}^{{n}_{E}} + {C}^{{n}_{E}}}$$

where Iₘₐₓ and Eₘₐₓ are the respective maximum percent current inhibition or enhancement, IC₅₀ and EC₅₀ are the drug concentrations C producing respective median inhibitory or enhancement of current, and m and WE are the respective Hill coefficients for the inhibitory and enhancing response curves. The methyl salicylate effect on GABA_{A} receptors was described by both inhibitory and enhancement Hill equations terms. All other drug-channel interactions were only inhibitory.

FIG. 5 is a linear-log plot of the percent current change from baseline of GABA_{A} receptors exposed to L-carvone. Data are from 4-8 oocytes at each concentration. The solid line is the best fit to the Hill equation:

$$- 100 \frac{{C}^{1.3}}{( 7.2 \times 1 {0}^{- 5} ) + {C}^{1.3}}$$

where C is the L-carvone concentration.

FIG. 6 is a linear-log plot of the percent current change from baseline of GABA_{A} receptors exposed to methyl salicylate. Data are from 4-10 oocytes at each concentration. The solid line is the best fit to the Hill equation:

$$- 73 \frac{{C}^{2.5}}{( 4.0 \times 1 {0}^{- 18} ) + {C}^{2.5}} + 223 \frac{{C}^{0.21}}{0.33 + {C}^{0.21}}$$

where C is the methyl salicylate concentration.

FIG. 7 is a linear-log plot of the percent current change from baseline of NMDA receptors exposed to L-carvone. Data are from 5-7 oocytes at each concentration. The solid line is the best fit to the Hill equation:

$$- 100 \frac{{C}^{0.8}}{( 8.0 \times 1 {0}^{- 3} ) + {C}^{0.8}}$$

where C is the L-carvone concentration.

FIG. 8 is a linear-log plot of the percent current change from baseline of NMDA receptors exposed to methyl salicylate. Data are from 5-7 oocytes at each concentration. The solid line is the best fit to the Hill equation:

$$- 100 \frac{{C}^{0.8}}{( 7.7 \times 1 {0}^{- 3} ) + {C}^{0.8}}$$

where C is the methyl salicylate concentration.

FIG. 9 is a linear-log plot of the percent current change from baseline of type-2 voltage-gated sodium channels exposed to L-carvone. Data are from 3-7 oocytes at each concentration. The solid line is the best fit to the Hill equation:

$$- 100 \frac{{C}^{0.9}}{( 1.5 \times 1 {0}^{- 3} ) + {C}^{0.9}}$$

where C is the L-carvone concentration.

FIG. 10 is a linear-log plot of the percent current change from baseline of type-2 voltage-gated sodium channels exposed to methyl salicylate. Data are from 6-9 oocytes at each concentration. The solid line is the best fit to the Hill equation:

$$- 100 \frac{{C}^{2.2}}{( 6.1 \times 1 {0}^{- 7} ) + {C}^{2.2}}$$

where C is the methyl salicylate concentration.

### DETAILED DESCRIPTION

Provided herein are compositions and methods for the administration of particular terpenoid and salicylate compounds for inducing anesthesia in human and non-human animal subjects for the purpose of inducing targeted anesthetic endpoints. Terpenoids, along with certain salicylates, are naturally-occurring and often aromatic compounds produced by plants and found in many foods. The present disclosure demonstrates that some terpenoids and salicylates can be prepared in a suitable vehicle and administered, e.g., by intravenous injection, to produce general anesthesia and other effects in mammals. At higher doses, these same compounds can be administered, e.g., by intravenous injection, to produce euthanasia. Sub-anesthetic doses can further be used to produce analgesia.

Advantageously, because the terpenoids and salicylates disclosed herein are found in human foods and are generally recognized as safe (GRAS), they can be used for anesthesia or analgesia in food-producing animals without the need for meat and milk withholding times. Accordingly, food-producing animals euthanized by administration of these agents can be subsequently consumed by humans or animals without injury. In addition, low toxicity and high environmental decomposition properties of the compounds disclosed herein offer significant advantages over use of, for example, pentobarbital for euthanasia, since the latter does not break down during animal rendering, has the potential to contaminate the food web, and is a controlled substance of potential abuse.

### I. COMPOSITIONS

In one aspect, a composition, e.g., a pharmaceutical composition, is disclosed. The composition provides surprising improvements in environmental, food processing, and drug control features of veterinary and human health. For example, the composition can advantageously decrease ecological holdover associated with slower degrading alternative anesthetic agents, reduce withholding times associated with food animal drug metabolism, and provide a less addictive alternative to agents that are controlled substances. The composition includes one or more compounds, i.e., active compounds, that are each individually a terpenoid or a salicylate. As such, the composition can include compounds that occur naturally within edible food products. The amount of the one or more compounds in the composition is effective for inducing anesthesia in a subject. In some embodiments, the composition further includes a delivery vehicle.

### Compounds

In some embodiments, the composition includes one active compound that is a terpenoid or a salicylate. In some embodiments, the composition includes two or more of such compounds, e.g., three or more, four or more, five or more, six or more, seven or more, eight or more, nine or more, or ten or more. In some embodiments, the active compounds of the composition consist of one or more terpenoids. In some embodiments, the active compounds of the composition consist of one or more salicylates. In some embodiments, the active compounds of the composition include at least one terpenoid and at least one salicylate.

As used herein, the term “terpenoid” refers to an organic chemical derived from the 5-carbon compound isoprene and typically containing additional functional groups, e.g., oxygen-containing functional groups. Each terpenoid compound of the provided composition, when the composition includes one or more terpenoids, can independently be an amphipathic terpenoid. In some embodiments, the active compounds of the composition consist of one or more amphipathic terpenoids. Each terpenoid compound of the provided composition, when the composition includes one or more terpenoids, can independently have or be derived from one, two, three, four, five, six, seven, eight, or more than eight isoprene units. In terms of lower limits, each terpenoid compound of the composition can independently have or be derived from one or more isoprene units, e.g., two or more isoprene units, three or more isoprene units, four or more isoprene units, five or more isoprene units, six or more isoprene units, seven or more isoprene units, or eight or more isoprene units. In terms of upper limits, each terpenoid compound of the composition can independently have or be derived from eight or fewer isoprene units, e.g., seven or fewer isoprene units, six or fewer isoprene units, five or fewer isoprene units, four or fewer isoprene units, three or fewer isoprene units, or two or fewer isoprene units.

In some embodiments, the provided composition includes a hemiterpenoid, i.e., a terpenoid having or being derived from one isoprene unit. In some embodiments, each terpenoid of the composition, when the composition includes one or more terpenoids, is a hemiterpenoid. The composition can include, for example, prenol (CAS #556-82-1). As used herein, the abbreviation “CAS #” refers to a Chemical Abstracts Service registry number.

In some embodiments, the provided composition includes a monoterpenoid, i.e., a terpenoid having or being derived from two isoprene units. In some embodiments, each terpenoid of the composition, when the composition includes one or more terpenoids, is a monoterpenoid. The composition can include, for example, 1-8, cineole (CAS #470-82-6), 4-allylphenyl acetate (CAS #61499-22-7), citral (CAS #5392-40-5), E-citral (CAS #141-27-5), Z-citral (CAS #106-26-3), pinene (CAS #473-55-2, (+)-α-pinene (CAS #80-56-8), (+)-β-pinene (CAS #127-91-3), (−)-β-pinene (CAS #18172-67-3), (+)-α-pinene (CAS #7785-70-8), (−)-α-pinene (CAS #7785-26-4), (+)-terpinen-4-ol (CAS #562-74-3), (+)-terpinen-4-ol (CAS #2438-10-0), (−)-terpinen-4-ol (CAS #20126-76-5), (+)-α-terpineol (CAS #98-55-5), (−)-α-terpineol (CAS #10482-56-1), (+)-α-terpineol (CAS #7785-53-7), β-terpineol (CAS #138-87-4), γ-terpineol (CAS #586-81-2), terpinolene (CAS #586-62-9), α-linalool (CAS #598-07-2), (CAS #78-70-6), α-myrcene (CAS #1686-30-2), β-myrcene (CAS #123-35-3), β-linalool phellandrene (CAS #1329-99-3), (+)-α-phellandrene (CAS #99-83-2), (+)-α-phellandrene (CAS #2243-33-6), (−)-α-phellandrene (CAS #4221-98-1), (+)-β-phellandrene (CAS #555-10-2), (+)-β-phellandrene (CAS #6153-16-8), (−)-β-phellandrene (CAS #6153-17-9), camphor (CAS #76-22-2), L-(−)-carvone (CAS #6485-40-1), D-(+)-carvone (CAS #2244-16-8, (+)-Carvone (CAS #99-49-0), cis-dihydro-α-terpineol (CAS #7322-63-6), cis-β-terpineol

(CAS #7299-40-3), (+)-β-citronellol (CAS #106-22-9), (−)-β-citronellol (CAS #7540-51-4), (−)-α-citronellol (CAS #6812-78-8), (+)-β-citronellol (CAS #1117-61-9), (+)-trans-carveol (CAS #1197-07-5), (+)-trans-carveol (CAS #18383-51-2), (+)-cis-carveol (CAS #7632-16-8), (−)-cis-carveol (CAS #2102-59-2), (−)-trans-carveol (CAS #2102-58-1), (+)-cis-carveol (CAS #1197-06-4), γ-terpinene (CAS #99-85-4), α-terpinene (CAS #99-86-5), β-terpinene (CAS #99-84-3), β-geraniol (CAS #106-24-1), α-geraniol (CAS #14459-10-0), (−)-germacrene D (CAS #23986-74-5), (+)-germacrene D (CAS #68005-97-0), (+)-germacrene A (CAS #28028-64-0), (+)-isomenthone (CAS #491-07-6), (−)-isomenthone (CAS #18309-28-9), (+)-isomenthone (CAS #1196-31-2), (+)-limonene (CAS #138-86-3), (S)-(−)-limonene (CAS #5989-54-8), (R)-(+)-limonene5989-27-5, (+)-linalyl acetate (CAS #115-95-7), S-(+)-linalyl acetate (CAS #51685-40-6), R-(−)-linalyl acetate (CAS #16509-46-9), (+)-menthol (CAS #15356-60-2), (−)-menthol (CAS #2216-51-5), menthol (CAS #1490-04-6), (+)-menthol (CAS #89-78-1), (+)-menthone (CAS #89-80-5), (−)-menthone (CAS #14073-97-3), (+)-menthone (CAS #3391-87-5), β-myrcene (CAS #123-35-3), α-myrcene (CAS #1686-30-2), m-cymene (CAS #535-77-3), o-cymene (CAS #527-84-4), p-cymene (CAS #99-87-6), (R)-(+)-pulegone (CAS #89-82-7), (+)-pulegone (CAS #15932-80-6), (S)-pulegone

(CAS #3391-90-0), m-thymol (CAS #89-83-8), o-thymol (CAS #3228-04-4), p-thymol (CAS #3228-02-2), 2-methoxy-4-vinyl phenol (CAS #7786-61-0), homovanillyl alcohol (CAS #2380-78-1), vanillyl alcohol (CAS #498-00-0), vanillin (CAS #121-33-5), eugenol (CAS #97-53-0), guaiacol (CAS #90-05-1), or a combination thereof. In some embodiments, the active compounds of the composition comprise L-carvone. In some embodiments, the active compound of the composition consists of L-carvone.

In some embodiments, the provided composition includes a sesquiterpenoid, i.e., a terpenoid having or being derived from three isoprene units. In some embodiments, each terpenoid of the composition, when the composition includes one or more terpenoids, is a sesquiterpenoid. The composition can include, for example, humolene (CAS #26472-41-3), α-calacorene (CAS #21391-99-1), β-calacorene (CAS #50277-34-4), α-caryophyllene (CAS #6753-98-6), β-caryophyllene (CAS #87-44-5), selinene (CAS #27104-12-7), (+)-α-selinene (CAS #64474-07-3), (−)-α-selinene (CAS #473-13-2), (+)-β-selinene (CAS #17066-67-0), (−)-β-selinene (CAS #473-12-1), (+)-bicyclogermacrene (CAS #100762-45-6), (+)-bicyclogermacrene (CAS #24703-35-3), (−)-bicyclogermacrene (CAS #67650-49-1), cadalene (CAS #483-78-3), (−)-8-cadinol (CAS #19435-97-3), (+)-8-cadinol (CAS #39864-11-4), (−)-β-caryophyllene (CAS #87-44-5), (+)-β-caryophyllene (CAS #10579-93-8), (+)-9-epi-β-caryophyllene (CAS #68832-35-9), (+)-β-elemene (CAS #33880-83-0), (−)-γ-elemene (CAS #29873-99-2), 8-elemene (CAS #20307-84-0), elemene (CAS #11029-06-4), (+)-α-elemene (CAS #5951-67-7), (−)-β-elemene (CAS #515-13-9), trans-α-bergamotene

(CAS #13474-59-4), cis-α-bergamotene (CAS #18252-46-5), trans-β-bergamotene (CAS #15438-94-5), cis-β-bergamotene (CAS #15438-93-4), bergamotene (CAS #6895-56-3), (+)-valencene (CAS #4630-07-3), (−)-valencene (CAS #724783-68-0), (+)-valencene (CAS #24741-64-8), (+)-cis-calamenene (CAS #72937-55-4), (+)-cis-calamenene (CAS #22339-23-7), (−)-cis-calamenene (CAS #483-77-2), (−)-trans-calamenene (CAS #35943-92-1), (+)-trans-calamenene (CAS #40772-39-2), or a combination thereof.

As used herein, the term “salicylate” refers to an organic chemical derived from the compound salicylic acid. A salicylate can be, for example, an ester of salicylic acid. In some embodiments, the active compounds of the provided composition comprise one or more esters of salicylic acid. In some embodiments, the active compounds of the provided composition consist of one or more esters of salicylic acid. In some embodiments, the active compounds of the composition comprise methyl salicylate. In some embodiments, the active compound of the composition consists of methyl salicylate. In some embodiments, the active compounds of the composition comprise ethyl salicylate. In some embodiments, the active compound of the composition consists of ethyl salicylate. In some embodiments, the active compounds of the composition comprise benzyl salicylate. In some embodiments, the active compound of the composition consists of benzyl salicylate. In some embodiments, the active compounds of the composition comprise methyl salicylate and ethyl salicylate. In some embodiments, the active compounds of the composition consist of methyl salicylate and ethyl salicylate. In some embodiments, the active compounds of the composition comprise methyl salicylate and benzyl salicylate. In some embodiments, the active compounds of the composition consist of methyl salicylate and benzyl salicylate. In some embodiments, the active compounds of the composition comprise ethyl salicylate and benzyl salicylate. In some embodiments, the active compounds of the composition consist of ethyl salicylate and benzyl salicylate. In some embodiments, the active compounds of the composition comprise methyl salicylate, ethyl salicylate, and benzyl salicylate. In some embodiments, the active compounds of the composition consist of methyl salicylate, ethyl salicylate, and benzyl salicylate. The methyl, ethyl, or benzyl salicylate can be a derivative such as, for example, 2-methoxy-methyl salicylate, 6-hydroxy-methyl salicylate, or the like.

In some embodiments, the provided composition includes at least one active compound that is a Type II voltage-gated sodium channel (Naᵥ1.2) antagonist. In some embodiments, each active compound of the composition is a Type II voltage-gated sodium channel antagonist. The Type II voltage-gated sodium channel plays a role in transmission of nociceptive signals, and other well-known analgesic drugs such as lidocaine are Type II voltage-gated sodium channel antagonists.

In some embodiments, the provided composition includes at least one active compound that is an N-methyl-D-aspartate (NMDA) receptor antagonists. In some embodiments, each active compound of the composition is an N-methyl-D-aspartate receptor antagonist. The N-methyl-D-aspartate receptor plays a role in transmission of nociceptive signals, and other well-known analgesic drugs such as ketamine are N-methyl-D-aspartate antagonists.

In some embodiments, the provided composition includes at least one active compound that is a γ-aminobutyric acid type A (GABA_{A}) receptor agonist. In some embodiments, each active compound of the composition is a GABA_{A} receptor agonist.

In some embodiments, the provided composition includes at least one active compound that is both a Type II voltage-gated sodium channel antagonist and an N-methyl-D-aspartate receptor antagonist. In some embodiments, each active compound of the composition is both a Type II voltage-gated sodium channel antagonist and an N-methyl-D-aspartate receptor antagonist. L-carvone and methyl salicylate, each of which is described above, are each independently both a Type II voltage-gated sodium channel antagonist and an N-methyl-D-aspartate receptor antagonist.

### Delivery Vehicles

As used herein, the term “delivery vehicle” refers to a component of a composition, e.g., a pharmaceutical composition, that aids in the delivery of a compound, e.g., an active compound, of the composition into and/or within the body of a subject. A delivery vehicle can be one that aids in the delivery of a compound to one or more particular organs in the body of a subject. A delivery vehicle can be one that aids in the delivery of a compound across one or more particular membranes or other barriers within the body of a subject. A delivery vehicle, when present in a composition, is typically present in an effective amount, i.e., an amount that increases the delivery of a compound to the body of a patient, to one or more targeted organs of the patient, or across one or more targeted barriers within the patient. Delivery vehicles are typically selected to not interfere with the biological activities of the compounds to be delivered within the body of the subject.

In some embodiments, the provided composition includes one or more delivery vehicles, e.g., two or more delivery vehicles, three or more delivery vehicles, four or more delivery vehicles, five or more delivery vehicles, six or more delivery vehicles, seven or more delivery vehicles, eight or more delivery vehicles, nine or more delivery vehicles, or ten or more delivery vehicles. In some embodiments, the one or more delivery vehicles of the composition include a lipid emulsion. In some embodiments, the one or more delivery vehicles consist of a lipid emulsion. In some embodiments, the one or more delivery vehicles include one or more oligosaccharides. In some embodiments, the one or more delivery vehicles consist of one or more oligosaccharides. In some embodiments, the oligosaccharides include one or more cyclic oligosaccharides such as cyclodextrins. In some embodiments, each oligosaccharide is independently a cyclic oligosaccharide such as a cyclodextrin. In some embodiments, the one or more delivery vehicles of the composition include one or more liposomes. In some embodiments, the one or more delivery vehicles consist of one or more liposomes. In some embodiments, the one or more delivery vehicles of the composition include a microemulsion. In some embodiments, the one or more delivery vehicles consist of a microemulsion. In some embodiments, the one or more delivery vehicles include one or more surfactants. In some embodiments, the one or more delivery vehicles consist of one or more surfactants. In some embodiments, the one or more delivery vehicles include glycerol. In some embodiments, the one or more delivery vehicles consist of glycerol. In some embodiments, the one or more delivery vehicles include one or more organic solvents. In some embodiments, the one or more delivery vehicles consist of one or more organic solvents. In some embodiments, the one or more organic solvents include dimethyl sulfoxide (DMSO). In some embodiments, the one or more organic solvents consist of DMSO.

### Other Composition Ingredients

The provided composition can further include additional ingredients, such as a pharmaceutically acceptable carrier. As used herein, the term “pharmaceutically acceptable carrier” refers to a material which does not abrogate the biological activity of properties of the active compounds disclosed herein, and which is involved in carrying or transporting the compounds within or to the subject. The pharmaceutically acceptable carrier can include, for example, a liquid or solid filler, stabilizer, dispersing agent, suspending agent, diluent, excipient, thickening agent, or a combination thereof. Each carrier must be “acceptable” in the sense of being compatible with the other ingredients of the composition, including the active compounds, and not injurious to the subject. Other examples of materials that can serve as pharmaceutically acceptable carriers include sugars, such as lactose, glucose and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound of the invention, and are physiologically acceptable to the subject.

Supplementary active compounds can also be incorporated into the compositions. Other additional ingredients that can be included in the pharmaceutical compositions used in the practice of the invention are known in the art and described, for example in Remington: The Science and Practice of Pharmacy (Remington: The Science & Practice of Pharmacy), 21^{st} Edition, 2011, Pharmaceutical Press; and Ansel's Pharmaceutical Dosage Forms and Drug Delivery Systems, Allen, et al., eds., 9th Edition, 2010, Lippincott Williams & Wilkins, which are incorporated herein by reference.

### Subjects

The composition disclosed herein is suitable use inducing anesthesia in a variety of different subjects. As used herein, the term “subject” refers to a vertebrate, and preferably to a mammal. Mammalian subjects for which the provided composition is suitable include, but are not limited to, mice, rats, simians, humans, farm animals, sport animals, and pets. The composition provides surprising improvements for inducement of anesthesia in farm or livestock animals, such as animals used for milk, meat, or wool production. For example, the composition can advantageously decrease milk and meat withholding times associated with food animal drug metabolism. In some embodiments, the subject is a non-human animal requiring anesthesia while undergoing a surgical or other medical procedure. In some embodiments, the subject is a non-human animal being euthanized with the anesthetic composition.

In some embodiments, the subject is human. In some embodiments, the subject is male. In some embodiments, the subject is female. In some embodiments, the subject is an adult. In some embodiments, the subject is an adolescent. In some embodiments, the subject is a child. In some embodiments, the subject is above 10 years of age, e.g., above 20 years of age, above 30 years of age, above 40 years of age, above 50 years of age, above 60 years of age, above 70 years of age, or above 80 years of age. In some embodiments, the subject is less than 80 years of age, e.g., less than 70 years of age, less than 60 years of age, less than 50 years of age, less than 40 years of age, less than 30 years of age, less than 20 years of age, or less than 10 years of age. In some embodiments, the subject is a human undergoing a surgical or other medical procedure and requiring temporary unconsciousness and/or immobility. In some embodiments, the subject is a human undergoing a surgical or other medical procedure and requiring a reduction in anxiety or pain or discomfort awareness.

### Anesthesia

As used herein, the term “anesthesia” refers to a state of controlled, temporary loss of sensation or awareness, that is generally induced for medical purposes. Anesthesia refers to conditions that include one or more of analgesia, e.g., relief from or prevention of pain; paralysis, e.g., muscle relaxation; amnesia, e.g., loss of memory; and unconsciousness. Anesthesia can enable the painless performance of medical procedures that would otherwise cause severe or intolerable pain to a non-anesthetized patient, or would otherwise be technically unfeasible. General anesthesia suppresses central nervous system activity and results in unconsciousness and total lack of sensation. Sedation through anesthesia suppresses the central nervous system to a lesser degree, inhibiting both anxiety and creation of long-term memories without resulting in unconsciousness. Regional and local anesthesia blocks transmission of nerve impulses from a specific part of the body.

The compositions disclosed herein are suitable for inducing anesthesia for a variety of different goals or endpoints. In some embodiments, the endpoint of the anesthesia includes analgesia. In some embodiments, the endpoint of the anesthesia consists of analgesia. In some embodiments, the endpoint of the anesthesia includes general anesthesia. In some embodiments, the endpoint of the anesthesia consists of general anesthesia. In some embodiments, the endpoint of the anesthesia does not include general anesthesia. In some embodiments, the endpoint of the anesthesia includes euthanasia, e.g., the rapid euthanasia of veterinary species of non-human animals. In some embodiments, the endpoint of the anesthesia consists of euthanasia. In some embodiments, the endpoint of the anesthesia includes tranquilization, e.g., tranquilization in the absence of general anesthesia. In some embodiments, the endpoint of the anesthesia consists of tranquilization. In some embodiments, the endpoint of the anesthesia includes sedation. In some embodiments, the endpoint of the anesthesia consists of sedation. In some embodiments, the endpoint of the anesthesia includes amnesia. In some embodiments, the endpoint of the anesthesia consists of amnesia. In some embodiments, the endpoint of the anesthesia includes a hypnotic state. In some embodiments, the endpoint of the anesthesia consists of hypnotic state. In some embodiments, the endpoint of the anesthesia includes insensitivity to noxious stimulation. In some embodiments, the endpoint of the anesthesia consists of insensitivity to noxious stimulation. In some embodiments, the endpoint of the anesthesia includes a combination of two or more of the above endpoint conditions.

### II. METHODS

In another aspect, a method of inducing anesthesia is disclosed. The method includes administering to a subject any of the compositions or compounds disclosed herein and described in further detail above. As the method uses the provided compositions and compounds, the method provides similar surprising improvements in environmental, food processing, and drug control features of veterinary and human health. For example, the method can advantageously decrease ecological holdover associated with slower degrading alternative anesthetic agents, reduce withholding times associated with food animal drug metabolism, and provide a less addictive alternative to agents that are controlled substances. The amount of the compound administered to the subject in the method is an amount effective for inducing anesthesia to the subject.

### Administering

As used herein, the term “administering” includes any mode of delivery suitable for using an effective amount of the compounds or compositions disclosed herein to induce anesthesia in a subject with the provided method. In some embodiments, the administering is via injection. In some embodiments, the injection is intravenous. In some embodiments, the injection is intramuscular. In some embodiments, the injection is subcutaneous.

The administering can include the delivery of the provided compounds or compositions in a discrete dose, a continuous dose, or a semi-continuous or stepwise dose. The administering can include the delivery of a single dose or multiple doses. In some embodiments, the administering is adjusted as needed based on the response of the subject to the administering, and the subject's transition to the anesthetic endpoint. The adjustment of the administering can depend on, for example, measurements of one or more of the pulse, blood pressure, breathing rate, or blood oxygen levels or other blood chemistries of the subject. In some embodiments, the provided method further includes comparing the determined level of one or more of such physiological parameters of the subject to predetermined reference levels of the physiological parameters.

### Effective Amounts

As used herein, the term “effective amount” refers to the amount of one or more compounds necessary to bring about the desired result, e.g., an amount sufficient to result in a desired anesthetic endpoint. The effective amount of the administered compound for inducing anesthesia in a subject using the method disclosed herein can be determined by such considerations as may be known to those of skill in the art. The effective amount can vary depending on one or more considerations. For example, the effective amount can depend on the particular compound selected; the species, gender, age, and/or medical condition of the subject; the desired one or more endpoints of the anesthesia; the dosage regimen to be followed; the presence of additional active compounds, delivery vehicles, or other components present in the administered composition or formulation; the choice or delivery method used for the administering; or a combination thereof.

Determination of an effective amount is well within the capability of those skilled in the art, especially in light of the detailed disclosure provided herein. Generally, an efficacious or effective amount of a combination of one or more anesthetic agents is determined by first administering a low dose or small amount of the anesthetic, and then incrementally increasing the administered dose or dosages, adding a second or third medication as needed, until a desired effect is observed in the treated subject with minimal or no toxic side effects. Applicable methods for determining an appropriate dose and dosing schedule for administration of anesthetics are described, for example, in Goodman and Gilman's The Pharmacological Basis of Therapeutics, 12th Edition, 2010; Physicians' Desk Reference (PDR); Remington: The Science and Practice of Pharmacy (Remington: The Science & Practice of Pharmacy), 21st Edition, 2011, Pharmaceutical Press; Ansel's Pharmaceutical Dosage Forms and Drug Delivery Systems, Allen, et al., eds., 9th Edition, 2010, Lippincott Williams & Wilkins; Martindale: The Complete Drug Reference, Sweetman, 2005, London: Pharmaceutical Press.; and Martindale, Martindale: The Extra Pharmacopoeia, 31st Edition., 1996, Amer Pharmaceutical Assn, each of which is incorporated herein by reference.

The effective amount of the compound for administration with the provided method can be selected in terms of a dosage. The dosage can be expressed in terms of the amount of compound administered divide by the body mass of the subject to whom the compound is administered. Standard units for dosage can be, for example, milligram of administered compound per kilogram of subject body mass. The effective amount of administered compound can include a dosage that is, for example, between 0.02 mg/kg and 2 mg/kg, e.g., between 0.02 mg/kg and 0.032 mg/kg, between 0.032 mg/kg and 0.5 mg/kg, between 0.05 mg/kg and 0.8 mg/kg, between 0.08 mg/kg and 1.3 mg/kg, or between 0.13 mg/kg and 2 mg/kg. In terms of upper limits, the dosage can be less than 2 mg/kg, e.g., less than 1.3 mg/kg, less than 0.8 mg/kg, less than 0.5 mg/kg, less than 0.32 mg/kg, less than 0.2 mg/kg, less than 0.13 mg/kg, less than 0.08 mg/kg, less than 0.05 mg/kg, or less than 0.032 mg/kg. In terms of lower limits, the dosage can be at least 0.02 mg/kg, e.g., at least 0.032 mg/kg, at least 0.05 mg/kg, at least 0.08 mg/kg, at least 0.13 mg/kg, at least 0.2 mg/kg, at least 0.32 mg/kg, at least 0.5 mg/kg, at least 0.8 mg/kg, or at least 1.3 mg/kg. Lower dosages, e.g., less than or equal to 0.02 mg/kg, are also contemplated. In some embodiments, the desired endpoint of the anesthesia includes analgesia, and the effective amount of compound to be administered resulting in this endpoint is within any of the above ranges of dosages less than or equal to 2 mg/kg.

In some embodiments, the effective amount of administered compound includes a dosage that is, for example, from 2 mg/kg to 4 mg/kg, e.g., from 2 mg/kg to 3.2 mg/kg, from 2.2 mg/kg to 3.4 mg/kg, from 2.4 mg/kg to 3.6 mg/kg, from 2.6 mg/kg to 3.8 mg/kg, or from 2.8 mg/kg to 4 mg·kg. In terms of upper limits, the dosage can be less than 4 mg/kg, e.g., less than 3.8 mg/kg, less than 3.6 mg/kg, less than 3.4 mg/kg, less than 3.2 mg/kg, less than 3 mg/kg, less than 2.8 mg/kg, less than 2.6 mg/kg, less than 2.4 mg/kg, or less than 2.2 mg/kg. In terms of lower limits, the dosage can be greater than 2 mg/kg, e.g., greater than 2.2 mg/kg, greater than 2.4 mg/kg, greater than 2.6 mg/kg, greater than 2.8 mg/kg, greater than 3 mg/kg, greater than 3.2 mg/kg, greater than 3.4 mg/kg, or greater than 3.6 mg/kg. In some embodiments, the desired endpoint of the anesthesia includes general anesthesia, and the effective amount of compound to be administered resulting in this endpoint is within any of the above ranges of dosages from 2 mg/kg to 4 mg/kg.

In some embodiments, the effective amount of administered compound includes a dosage that is, for example, between 4 mg/kg and 100 mg/kg, e.g., between 4 mg/kg and 28 mg/kg, between 5.5 mg/kg and 38 mg/kg, between 7.6 g/kg and 53 mg/kg, between 11 mg/kg and 72 mg/kg, or between 14 mg/kg and 100 mg/kg. In terms of upper limits, the dosage can be less than 100 mg/kg, e.g., less than 72 mg/kg, less than 53 mg/kg, less than 38 mg/kg, less than 28 mg/kg, less than 20 mg/kg, less than 14 mg/kg, less than 11 mg/kg, less than 7.6 mg/kg, less than 5.5 mg/kg. In terms of lower limits, the dosage can be greater than 4 mg/kg, e.g., greater than 5.5 mg/kg, greater than 7.6 mg/kg, greater than 11 mg/kg, greater than 14 mg/kg, greater than 20 mg/kg, greater than 28 mg/kg, greater than 38 mg/kg, greater than 53 mg/kg, or greater than 72 mg/kg. Higher dosages, e.g., greater than or equal to 100 mg/kg, are also contemplated. In some embodiments, the desired endpoint of the anesthesia includes euthanasia, and the effective amount of compound to be administered resulting in this endpoint is within any of the above ranges of dosages greater than or equal to 4 mg/kg.

### EMBODIMENTS

The following embodiments are contemplated. All combinations of features and embodiment are contemplated.

Embodiment 1: A method of inducing anesthesia in a subject, the method comprising administering to the subject an amount of a compound selected from the group consisting of a terpenoid, a salicylate, and a combination thereof, wherein the amount is effective for inducing anesthesia in the subject.

Embodiment 2: An embodiment of embodiment 1, wherein the compound is a Type II voltage-gated sodium channel antagonist.

Embodiment 3: An embodiment of embodiment 1 or 2 wherein the compound is an N-methyl-D-aspartate receptor antagonist.

Embodiment 4: An embodiment of any of the embodiments of embodiment 1-3, wherein the compound is a γ-aminobutyric-acid-A receptor agonist.

Embodiment 5: An embodiment of any of the embodiments of embodiment 1-4, wherein the compound is a terpenoid.

Embodiment 6: An embodiment of embodiment 5, wherein the terpenoid has only one isoprene unit.

Embodiment 7: An embodiment of embodiment 5, wherein the terpenoid has only two isoprene units.

Embodiment 8: An embodiment of embodiment 7, wherein the terpenoid is L-carvone.

Embodiment 9: An embodiment of embodiment 5, wherein the terpenoid has only three isoprene units.

Embodiment 10: An embodiment of any of the embodiments of embodiment 1-3, wherein the compound is a salicylate.

Embodiment 11: An embodiment of embodiment 10, wherein the salicylate is an ester of salicylic acid.

Embodiment 12: An embodiment of embodiment 11, wherein the salicylate is selected from the group consisting of methyl salicylate, ethyl salicylate, and benzyl salicylate.

Embodiment 13: An embodiment of any of the embodiments of embodiment 1-12, wherein the administering is via injection.

Embodiment 14: An embodiment of embodiment 13, wherein the injection is intravenous, intraperitoneal, intramuscular, or subcutaneous.

Embodiment 15: An embodiment of any of the embodiments of embodiment 1-14, wherein the subject is a mammal.

Embodiment 16: An embodiment of any of the embodiments of embodiment 1-15, wherein the anesthesia has an endpoint comprising analgesia, tranquilization, sedation, amnesia, a hypnotic state, a state of insensitivity to noxious stimulation, or a combination thereof.

Embodiment 17: An embodiment of any of the embodiments of embodiment 1-15, wherein the effective amount comprises a dosage less than or equal to 2 mg/kg.

Embodiment 18: An embodiment of embodiment 17, wherein the anesthesia has an endpoint comprising analgesia.

Embodiment 19: An embodiment of any of the embodiments of embodiment 1-15, wherein the effective amount comprises a dosage of from 2 mg/kg to 4 mg/kg.

Embodiment 20: An embodiment of embodiment 19, wherein the anesthesia is general anesthesia.

Embodiment 21: An embodiment of any of the embodiments of embodiment 1-15, wherein the effective amount comprises a dosage greater than or equal to 4 mg/kg.

Embodiment 22: An embodiment of embodiment 21, wherein the anesthesia has an endpoint comprising euthanasia.

Embodiment 23: An embodiment of any of the embodiments of embodiment 15-22, wherein the mammal is a livestock animal used for meat, milk, or wool production.

Embodiment 24: An embodiment of any of the embodiments of embodiment 15-22, wherein the mammal is a dog, cat, rabbit, rodent, horse, or other companion animal.

Embodiment 25: An embodiment of any of the embodiments of embodiment 15-20, wherein the mammal is a human.

Embodiment 26: An embodiment of any of the embodiments of embodiment 1-25, wherein the compound is formulated in a composition comprising a delivery vehicle.

Embodiment 27: An embodiment of embodiment 26, wherein the delivery vehicle comprises a lipid emulsion.

Embodiment 28: An embodiment of embodiment 26 or 27, wherein the delivery vehicle comprises a cyclodextrin.

Embodiment 29: A composition for inducing anesthesia in a subject, the composition comprising: an amount of a compound selected from the group consisting of a terpenoid, a salicylate, and a combination thereof, wherein the amount is effective for inducing anesthesia in the subject; and a delivery vehicle.

Embodiment 30: An embodiment of embodiment 29, wherein the compound is a Type II voltage-gated sodium channel antagonist.

Embodiment 31: An embodiment of embodiment 29 or 30 wherein the compound is a N-methyl-D-aspartate receptor antagonist.

Embodiment 32: An embodiment of any of the embodiments of embodiment 29-31, wherein the compound is a γ-aminobutyric-acid-A receptor agonist.

Embodiment 33: An embodiment of any of the embodiments of embodiment 29-32, wherein the compound is a terpenoid.

Embodiment 34: An embodiment of embodiment 33, wherein the terpenoid has only one isoprene unit.

Embodiment 35: An embodiment of embodiment 33, wherein the terpenoid has only two isoprene units.

Embodiment 36: An embodiment of embodiment 35, wherein the terpenoid is L-carvone.

Embodiment 37: An embodiment of embodiment 33, wherein the terpenoid has only three isoprene units.

Embodiment 38: An embodiment of any of the embodiments of embodiment 29-31, wherein the compound is a salicylate.

Embodiment 39: An embodiment of embodiment 38, wherein the salicylate is an ester of salicylic acid.

Embodiment 40: An embodiment of embodiment 39, wherein the salicylate is selected from the group consisting of methyl salicylate, ethyl salicylate, and benzyl salicylate.

Embodiment 41: An embodiment of any of the embodiments of embodiment 29-40, wherein the subject is a mammal.

Embodiment 42: An embodiment of any of the embodiments of embodiment 29-41, wherein the anesthesia has an endpoint comprising analgesia, tranquilization, sedation, amnesia, a hypnotic state, a state of insensitivity to noxious stimulation, or a combination thereof.

Embodiment 43: An embodiment of any of the embodiments of embodiment 29-41, wherein the effective amount comprises a dosage less than or equal to 2 mg/kg.

Embodiment 44: An embodiment of embodiment 43, wherein the anesthesia has an endpoint comprising analgesia.

Embodiment 45: An embodiment of any of the embodiments of embodiment 29-41, wherein the effective amount comprises a dosage of from 2 mg/kg to 4 mg/kg.

Embodiment 46: An embodiment of embodiment 45, wherein the anesthesia is general anesthesia.

Embodiment 47: An embodiment of any of the embodiments of embodiment 29-41, wherein the effective amount comprises a dosage greater than or equal to 4 mg/kg.

Embodiment 48: An embodiment of any of the embodiments of embodiment 47, wherein the anesthesia has an endpoint comprising euthanasia.

Embodiment 49: An embodiment of any of the embodiments of embodiment 41-48, wherein the mammal is a livestock animal used for meat, milk, or wool production.

Embodiment 50: An embodiment of any of the embodiments of embodiment 41-48, wherein the mammal is a dog, cat, rabbit, rodent, horse, or other companion animal.

Embodiment 51: An embodiment of any of the embodiments of embodiment 41-46, wherein the mammal is a human.

Embodiment 52: An embodiment of any of the embodiments of embodiment 29-51, wherein the delivery vehicle comprises a lipid emulsion.

Embodiment 53: An embodiment of any of the embodiments of embodiment 29-52, wherein the delivery vehicle comprises a cyclodextrin

### III. EXAMPLES

The present disclosure will be better understood in view of the following non-limiting examples. The following examples are intended for illustrative purposes only and do not limit in any way the scope of the present invention.

### Example 1. In Vitro Electrophysiology

Ovaries removed from sexually mature Xenopus laevis frogs were bluntly dissected to remove surrounding connective tissue and then digested with 0.2% type I collagenase in an oocyte Ringer's solution. Once free from thecal cells and connective tissue, individual oocytes were rinsed, sorted, and stored in a modified Barth's solution (R. Brosnan et al., Anesth. Analg. 103, (2006): 86; L. Yang, P. S. Milutinovic, R. J. Brosnan, E. I. Eger 2nd, & J. M. Sonner, Anesth. Analg. 105, (2007): 393; R. J. Brosnan, F. B. Fukushima, & T. L. Pham, Anaesth. Analg. 44, (2017): 577; A. Cenani, R. J. Brosnan, & H. K. Knych, Pharmacology 103, (2019): 10).

Heteromeric GABA_{A} receptors were expressed in oocytes by blind intranuclear injection of 1 ng DNA encoding the human al (GABRA1) and rat B2 (GABRB2) subunits in a 1:1 ratio. NMDA receptors were expressed by intracytoplasmic injection of 5 ng RNA encoding rat NR1 (GRIN1) and rat NR2a (GRIN2A) subunits in a 1:1 ratio. Nax 1.2 channels were expressed by injection of 5 ng RNA encoding the human SCN2A gene.

Two-electrode voltage clamp studies of GABA_{A} receptors were carried out in a 0.25-mL linear perfusion chamber. Perfusates were delivered by using a syringe pump at 1.5 mL/min using gastight glass syringes and polytetrafluoroethylene tubing. Oocytes were impaled by two 3 M KCl-filled 0.2-1 MΩ borosilicate glass electrodes (KG-33; King Precision Glass, Claremont, CA, USA) connected to separate headstages (Axon Instruments HS2A; Molecular Devices, San Jose, CA, USA) through which voltage was clamped at −80 mV and current was passed via a computer-controlled amplifier (Axon Instruments GeneClamp 500B; Molecular Devices). Oocytes were perfused with frog Ringer's (FR) solution containing 115 mM NaCl, 2.5 mM KCl, 1.8 mM CaCl₂), and 10 mM HEPES prepared from ACS-grade chemicals and 18.2 MΩ H2O, filtered and pH adjusted to 7.4. At 5-min intervals, the solution was switched to FR containing 50 μM GABA; this concentration produces a chloride current equal to 35-40% of a maximum GABA agonist response. After 30 s, the perfusate was switched back to FR solution. This process was repeated until a stable baseline response was achieved. Next, FR containing either L-carvone (99%; Sigma-Aldrich, St. Louis, MO, USA) or methyl salicylate (>99%; Sigma-Aldrich) at the test concentration was used to perfuse the oocyte for 1.5 min, and then the oocyte was perfused with FR+GABA solution containing this same drug and concentration for 30 s. Last, the solution was switched back to FR to wash out the drug for 5 min, and then FR+GABA was perfused for 30 s to confirm that the post-drug agonist response had returned to within 10% of the pre-drug response. Uninjected oocytes served as negative controls. Only one drug and concentration were studied per oocyte.

Studies of NMDA receptors were carried out using equipment identical to that described for GABA_{A} receptors. Transmembrane potential was clamped at −80 mV, and oocytes were perfused with a barium-containing FR solution (Ba-FR) which substituted equimolar BaCl₂ for calcium salts and contained 0.1 mM EGTA as a calcium chelator. After 5 min, the perfusate was changed for 30 s to barium FR containing 0.1 mM glutamate plus 0.01 mM glycine (Ba-FREG) as receptor agonists which produce an NMDA receptor current greater than 99% of the maximal response. Next, Ba-FR containing the study drug was perfused for 5 min and then switched to Ba-FREG containing the same test article for 30 s. The drug was then washed out with a 5-min perfusion of Ba-FR followed by a 30-s exposure to Ba-FREG to confirm post-drug currents had returned to within 10% of pre-drug values.

Studies of the Naᵥ1.2 channels were carried out Using the same equipment as above, oocytes were perfused with FR and clamped at −80 mV transmembrane potential. Every 2 min, a 6-s step potential to 0 mV was applied to open the channel; this was repeated until baseline responses stabilized. Next, FR containing the test drug was perfused for 2 min followed by another 6-s step voltage change to 0 mV. Finally, FR was perfused for 5 min to wash out the drug, and another 6-s step clamp to 0 mV was applied to confirm that post-drug currents had returned to within 10% of pre-drug values.

The change in whole cell peak current before and during agonist exposure (for GABA_{A} and NMDA receptors) or before and during the voltage step clamp (for Naᵥ1.2 channels) was measured in the tracings immediately before and during drug exposure. Percent change in current was calculated as follows:

$$\% Δ = \frac{{I}_{D} - {I}_{B}}{{I}_{B}} \times 100$$

where I_{B} is the baseline pre-drug current peak and ID is the drug current peak. For each drug and ion channel, data were fit to a Hill equation using nonlinear regression with sequential quadratic programming and bootstrap estimates of parameter standard errors (SPSS, v. 27; IBM, Armonk, NY, USA). In the model, maximum drug inhibition (Iₘₐₓ) was constrained to not be less than −100%. Initial parameter estimates were seeded with values based on visual inspection of respective dose-response curves in order to facilitate model convergence.

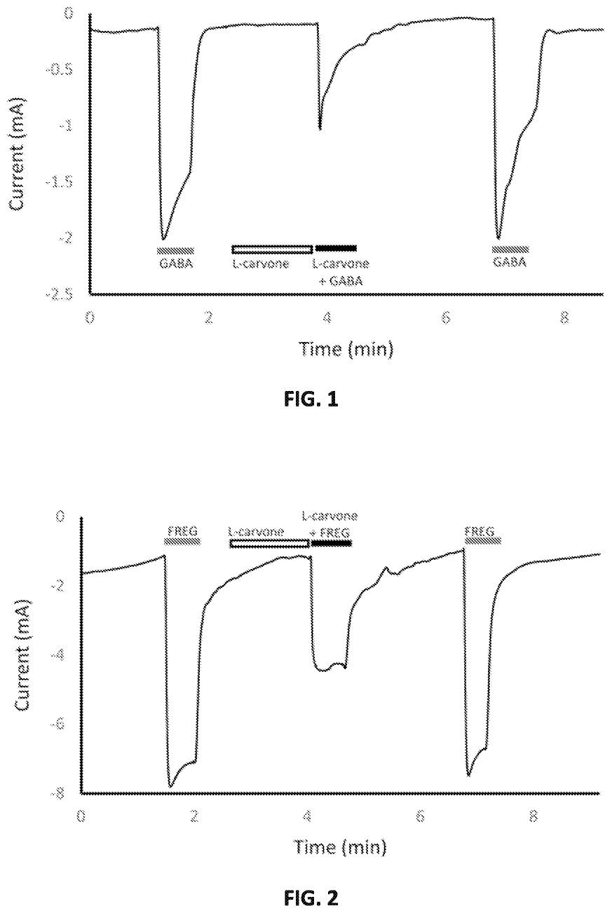

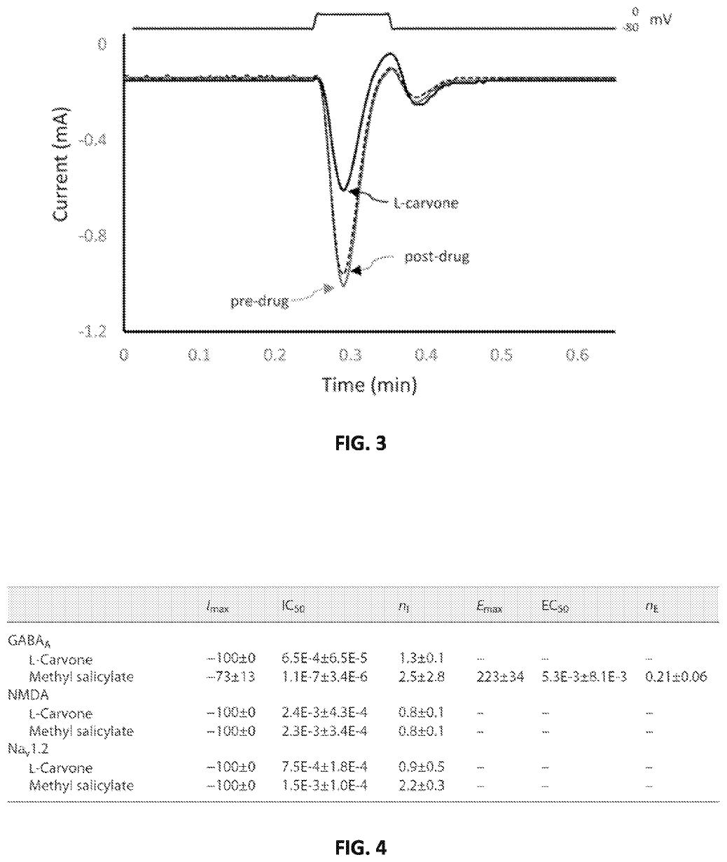

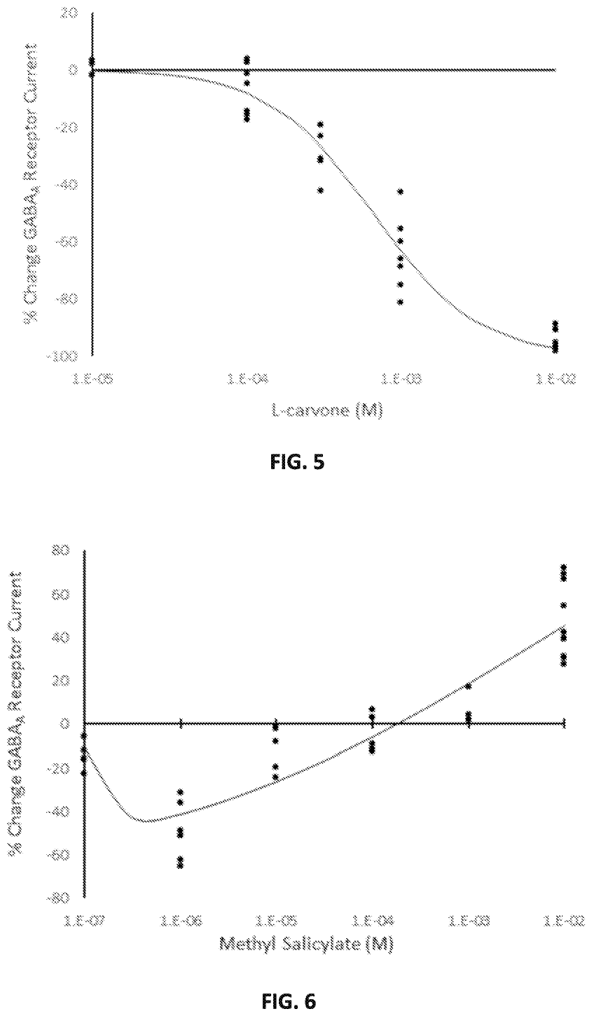

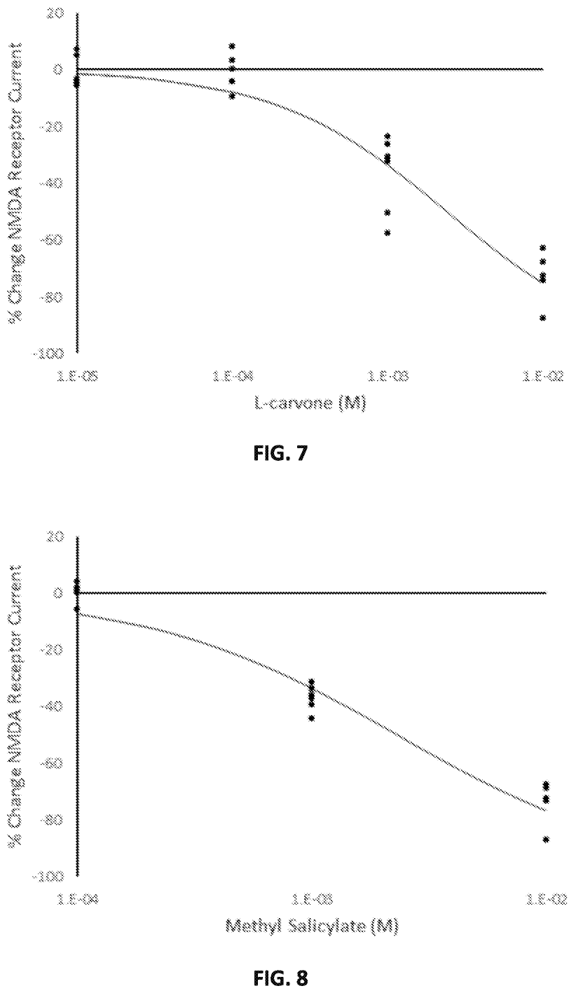

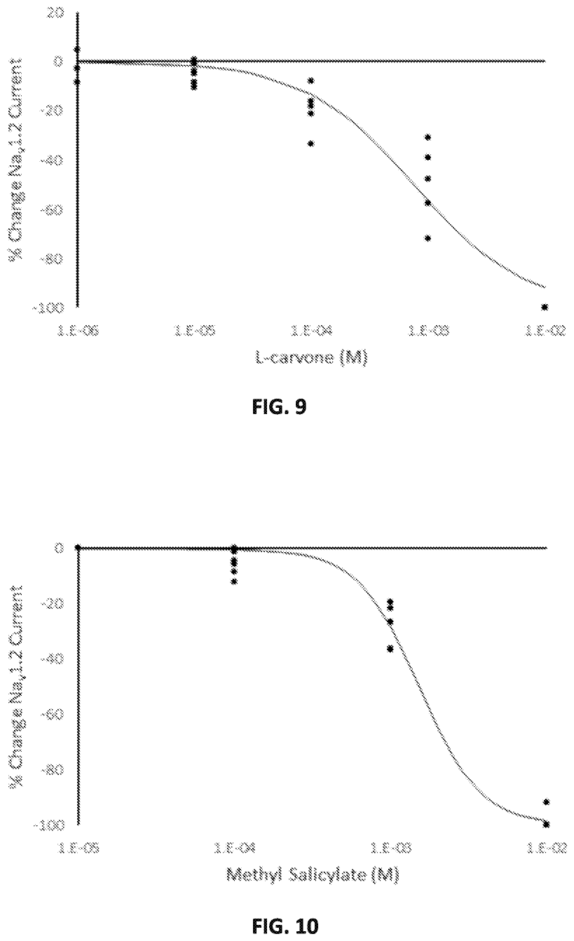

Sample tracings for GABA_{A} receptor, NMDA receptor, and Naᵥ1.2 channel experiments are shown in FIGS. 1-3. L-Carvone caused dose-dependent inhibition of all 3 ion channels (FIGS. 5, 7, and 9) with near or complete loss of channel currents at the 10 mM drug dose. Potency of L-carvone, as assessed by the median inhibitory concentration (IC50) for each channel (FIG. 4), was ranked as follows: GABA_{A}≥Naᵥ1.2>NMDA.

Methyl salicylate also dose-dependently inhibited NMDA receptors and Naᵥ1.2 channels (FIGS. 8 and 10) with the drug exhibiting 50% greater potency for the latter (FIG. 4). However, GABA_{A} receptors were potently inhibited by methyl salicylate (FIG. 6) with an IC50 of 0.11 μM indicative of a high-affinity receptor binding site (FIG. 4). At high concentrations, methyl salicylate binds a second site to potentiate GABA_{A} currents with a median effective concentration estimated at 5.3 mM, the weakest drug-receptor interaction observed in this Example.

Hill coefficient estimates for the dose-response models were close to 1 for all ion channels with L-carvone and for NMDA receptors with methyl salicylate (FIG. 4). This is consistent with drug-receptor binding independent of agonist binding (in the case of GABA_{A} and NMDA receptors) and either a single drug-protein binding site or noncooperative binding at multiple sites. The Naᵥ1.2 dose response curve for methyl salicylate was significantly greater than 1, suggesting cooperative binding that might occur from two or more drug molecules binding the channel at two or more interactive sites. In contrast, the Hill coefficient less than 1 for low-affinity methyl salicylate binding at the GABA_{A} receptor potentiation site indicates negative cooperative binding, possibly from interactions with methyl salicylate binding at the high-affinity GABA_{A} receptor inhibitory site.

### Example 2. In Vivo Anesthesia

Solutions of 10% (v/v) L-carvone or methyl salicylate were prepared sterilely in 20% INTRALIPID® (Fresenius Kabi, Lake Zurich, IL, USA). Lateral tail vein catheters were placed in twenty-one 69-day-old, male, Sprague Dawley rats weighing 291±17 (mean±SD) grams. Each rat received a single intravenous bolus injection of L-carvone solution (0.2 or 0.4 mL), methyl salicylate solution (0.1, 0.15, 0.2, or 0.4 mL), or a 1:1 mixture of the L-carvone and methyl salicylate solutions over 15 s. Because L-carvone density is 0.9673 g/cm³ at 25° C., 0.2 mL and 0.4 mL of solution contained 19 mg and 39 mg of L-carvone, respectively (E. E. Royals & S. E. Horne, J. Am. Chem. Soc. 73, (1951): 5850). The density of methyl salicylate is 1.1798 g/cm³ at 25° C.; hence, 0.2 mL and 0.4 mL of solution contained 24 mg and 47 mg of methyl salicylate, respectively (R. C. Glowaski & C. C. Lynch, J. Am. Chem. Soc. 55, (1933): 4051).

Spontaneous motor activity and loss-of-righting reflex was assessed at 5- to 10-s intervals, and nociception response to an alligator clamp applied to the tail was tested if the righting reflex was absent. Animals administered the lower doses of L-carvone, methyl salicylate, and the drug mixture were recovered and returned to the vivarium. Animals were weighed 24 and 48 h after the study and then euthanized for gross pathology examination.

Lateral tail veins were catheterized in eight, 70-day-old, male, Sprague Dawley rats weighing 309±6 g. A 10% solution of L-carvone or methyl salicylate in 20% INTRALIPID® was each administered IV to 8 rats (4 animals per drug treatment) by using a syringe pump at a rate of 12 mL/h. When rats lost their righting reflex and were unresponsive to a tail clamp, a midline laparotomy was performed, the abdominal aorta catheterized, and the rat exsanguinated. Blood was collected in a heparin tube and centrifuged; plasma was separated and stored in cryogenic tubes at −80° C. until analysis.

Serum calibrations were prepared by dilution of working standard solutions of methyl salicylate and L-carvone with drug-free rat serum (Innovative Research, Novi, MI, USA) to concentrations ranging from 0.05 to 500 μg/mL. Calibration curves and negative control samples were prepared fresh for each quantitative assay. In addition, quality control samples (drug-free rat serum fortified with analyte at 3-4 concentrations within the standard curve) were included as an additional check of accuracy.

Prior to analysis, 100 μL of each serum sample was diluted with 100 μL of acetonitrile (ACN):1 M acetic acid (9:1, v:v) and 50 μL of methanol to precipitate proteins. The samples were vortexed for 2 min to mix, refrigerated for 20 min, vortexed for an additional 1 min, centrifuged (4,300 rpm/3, 102 g) for 10 min at 4º C, and 10 μL injected into the liquid chromatography tandem mass spectrometry system.

Quantitative analysis was performed on an LTQ XL Orbitrap mass spectrometer (Thermo Scientific, San Jose, CA, USA) coupled with a Waters Acquity UPLC (Milford, MA, USA). The system was operated at a resolution of 15,000 (M/ΔM, at full width at half maximum of the mass peaks) using positive electrospray ionization (ESI[+]). Detection and quantification were conducted using full-scan accurate mass from 145 to 160 (m/z). The responses were plotted using a 20-ppm data window for methyl salicylate (mass-to-charge ratio 153.05443 [m/z]) and L-carvone (mass-to-charge ratio 151.11160 [m/z]). The spray voltage was 3,500 V, and the sheath and auxiliary gas was 45 and 20, respectively (arbitrary units). Chromatography employed an Accucore Vanquish C18+10 cm×2.1 mm 1.5 m column (Thermo Scientific) and a linear gradient of ACN in water with a constant 0.2% formic acid at a flow rate of 0.1 mL/min. The initial ACN concentration was held at 1% for 0.4 min and ramped to 99% over 9 min before re-equilibrating for 14 min at initial conditions.

The precision and accuracy of the assay was determined by assaying quality control samples in replicates (n=6). Accuracy was reported as percent nominal concentration and precision as percent relative standard deviation. For both methyl salicylate and L-carvone, all values were within 10% of the nominal concentration. The technique was optimized to provide limits of quantitation of 0.5 μg/mL and 0.05 μg/mL and limits of detection of approximately 0.1 μg/mL and 0.025 μg/mL for methyl salicylate and L-carvone, respectively.

In the pilot study IV bolus injections, 0.2 mL of the L-carvone emulsion (N=4 rats) resulted in transient excitement followed by loss-of-righting reflex in 40±6 s and loss of tail clamp response in 54±11 s. During this time, the respiratory rate was transiently decreased for approximately 10 s. Half of the rats exhibited whole-body muscle hypertonia for 15 s, after which muscles were relaxed. Recovery was rapid with righting reflex returning in 58±14 s and the rats walking and active in 60±13 s. Body weight over the next 2 days was within 1.9±1.4 percent of baseline, and no gross pathology findings were noted on examination of the heart, lung, liver, or kidney. Similar intravenous injection of 0.4 mL of L-carvone (N=4 rats) induced unconsciousness and flaccid muscle tone in 20±4 s and cardiopulmonary arrest in 35±18 s.

A 0.1 mL IV bolus of the methyl salicylate emulsion in a single rat caused loss- and return-of-righting reflex in 33 and 60 s, respectively, but never resulted in general anesthesia, as defined by immobility in response to a tail clamp. Increasing the methyl salicylate injection dose to 0.15 mL (N=4 rats) caused loss-of-righting in 35±5 s and anesthesia in 39±2 s, and both returned in 53±3 and 64±8 s after drug injection with normal ambulation without ataxia at 76±10 s. Following a 0.2 mL methyl salicylate emulsion injection (N=3 rats), loss-of-righting and general anesthesia occurred in 35±10 and 37±13 s, respectively. However, 1 rat exhibited cardiopulmonary arrest 35 s following this dose of methyl salicylate. The other 2 rats regained tail clamp and righting responses an average of 48 s and 58 s, respectively, and were ambulating normally 110 s following drug administration. For those rats receiving sublethal methyl salicylate doses, body weight was within 97-98% of baseline values for the next two days, and no gross abnormalities were found on necropsy 48 h after drug administration. Increasing the methyl salicylate emulsion dose to 0.4 mL (N=3 rats) resulted in unconsciousness in 20±5 s and cardiopulmonary arrest in all animals 31±sec after beginning injection.

Co-administration of 0.1 L-carvone emulsion plus 0.1 mL methyl salicylate emulsion (N=2 rats) produced almost identical signs over a similar time scale as the anesthetic doses of described for either L-carvone emulsion or methyl salicylate emulsion administered alone. Body weights for both animals over the next 2 days ranged between 98 and 101% of baseline, and necropsy examinations were unremarkable. As there was no obvious benefit to this combination over administration of only the single agent, the pharmacodynamics of L-carvone/methyl salicylate mixtures were not further evaluated.

Continuous 0.2 mL/min IV infusions of L-carvone emulsions produced muscle fasciculations and loss-of-righting reflex followed by tonic muscle contractions suggestive of seizure over 2-4 min after the start of injection. After this time, there was no response to toe pinch and tail clamp, and the animals were euthanized by exsanguination. Similar injection with methyl salicylate emulsions in rats caused transient excitement in half of the animals 30 s after injection. Loss-of-righting and absent tail clamp responses occurred within 3-4 min after start of injection, after which rats were euthanized by exsanguination. No tonic or clonic muscle activity was observed in any rat administered methyl salicylate infusions. The mean plasma concentration (±SEM) of L-carvone during IV infusion in rats (N=4) was 7.9±3.0 mM (N=4). This concentration of L-carvone corresponded to 96% inhibition of GABA_{A} receptors (FIG. 5), 71% inhibition of NMDA receptors (FIG. 5), and 90% inhibition of Naᵥ1.2 channels (FIG. 9) for in vitro electrophysiology studies. Methyl salicylate concentration during IV infusions in rats (N=4) was 2.7±0.4 mM. This methyl salicylate concentration was associated with 30% potentiation of GABA_{A} receptors (FIG. 6), 53% inhibition of NMDA receptors (FIG. 8), and 78% inhibition of Naᵥ1.2 channels (FIG. 10) in vitro.

In another experiment, a 10% (v/v) solution of methyl salicylate in INTRALIPID®, and a 10% (v/v) solution of L-carvone in INTRALIPID® were each tested in Sprague Dawley rats that each weighed approximately 300 g. Intravenous doses of 0.2 mL of the L-carvone mixture caused general anesthesia within 30 seconds as evidenced by a loss of righting reflex and a loss of tail clamp response. The general anesthesia effects lasted for 1 minute. Within 3 minutes after administration, the rat was awake and ambulatory. No evidence of hepatic, renal, cardiac, or pulmonary injury was noted on a gross necropsy performed 2 days later. When 0.4 mL of the L-carvone mixture was administered intravenously, anesthesia occurred within 45 seconds, apnea followed almost immediately, and cardiac arrest was confirmed 2 minutes after that.

Intravenous doses of 0.2 mL of the methyl salicylate mixture caused a loss of righting reflex within 25 seconds, with the effect lasting for 30 seconds. The rat was awake and ambulatory 40 seconds afterwards. No evidence of hepatic, renal, cardiac, or pulmonary injury was noted on a gross necropsy performed 2 days later. When 0.4 mL of the methyl salicylate mixture was administered intravenously, loss of righting occurred after 15 seconds, followed by loss of tail clamp response and apnea 5 seconds later. Cardiac arrest was confirmed less than 1 minute later.

A 50:50 mixture of the L-carvone-INTRALIPID® and methyl salicylate-INTRALIPID® preparation was also created. When 0.2 mL of this mixture was administered intravenously, righting reflex was lost by the rat within 30 seconds and was returned 40 seconds after drug administration. The rat was ambulatory 30 seconds later, and ataxia resolved within 30 seconds afterwards. No evidence of hepatic, renal, cardiac, or pulmonary injury was noted on a gross necropsy performed 2 days later. When 0.4 mL of the mixture was administered intravenously, righting reflex was lost by 30 seconds, and apnea occurred 5 seconds later. Cardiac arrest was confirmed less than 2 minutes after administration of the drug mixture.

These results demonstrate that 0.2% (v/v) of either drug injected intravenously briefly produces general anesthesia in 300-g to 315-g rats, with a continuous infusion of 0.2 mg/kg/min sufficient to maintain a deep plane of anesthesia. A dose that is doubled in concentration produces euthanasia. Additionally, a rat to which the agents were administered subcutaneously became noticeably sedate for a longer duration than observed with intravenous dosing. This finding suggests that analgesic, sedative, and anesthetic endpoints may be reached via other non-intravenous parenteral routes such as subcutaneous routes and intramuscular routes, likely at higher doses than those for intravenous administration.

Amphipathic compounds with sufficient molar water solubility can modulate anesthetic-sensitive ion channels such as GABA_{A} receptors, NMDA receptors, and Naᵥ1.2 channels via low-affinity interactions, often at hundreds of micromolar concentrations or higher. The mint compounds L-carvone and methyl salicylate are more soluble in water than the cutoff values for modulation of these 3 anesthetic-sensitive ion channels (R. J. Brosnan & T. L. Pham, BMC Pharmacol. Toxicl. 15, (2016): 13; R. J. Brosnan & T. L. Pham, BMC Pharmacol. Toxicol. (2014): 15). The Examples provided herein demonstrate that both mint compounds do indeed modulate GABA_{A} receptor, NMDA receptor, and Naᵥ1.2 channel currents at concentrations associated with general anesthesia in rats.

L-Carvone application to the sciatic nerve axon of frogs decreases compound action potential amplitude with an IC50 of 1.4 mM (S. Ohtsubo, T. Fujita, A. Matsushita, & E. Kumamoto, Pharmacol. Res. Perspect. 3, (2015): e00127). This is similar to the IC50 for human Naᵥ1.2 channels in the present study (FIG. 4). Given the central role of nodal Naᵥ1.2 channels for action potential propagation along nerve axons, results support a local anesthetic mechanism of drug action. L-Carvone also decreases compound action potentials in rat sciatic nerves, but at an IC50 of 8.7 mM (J. C. Goncalves, M. Alves Ade, A. E. de Araujo, J. S. Cruz, & D. A. Araujo, Eur. J. Pharmacol. 645, (2010): 108). This potency variability suggests significant species differences in drug-receptor binding affinity.

A previous 2-electrode voltage clamp study of human benzodiazepine-sensitive α1β2γ2s GABA_{A} receptors expressed in frog oocytes showed minimal response to L-carvone concentrations up to 0.12 mM (A. C. Hall et al., Eur. J. Pharmacol. 506, (2004): 9). In rat cerebral cortex cell cultures, 0.75 mML-carvone exerted a negative allosteric effect with GABA_{A}-induced benzodiazepine binding reduced by 36% (M. Sanchez-Borzone, L. Delgado-Marin, & D. A. Garcia, Chirality 26, (2014): 368). However, even greater GABA_{A} receptor modulation occurs at higher L-carvone concentrations. GABA_{A} receptor currents were almost entirely inhibited by 10 mML-carvone, the highest concentration studied here.

Mint oils have been used as immersion anesthetics in fish. Baths containing either 1.9-4.5 mML-carvone or 0.8-2.3 mM methyl salicylate caused anesthesia in carp with the highest doses associated with faster onset and longer recovery times (Z. Roohi & M. R. Imanpoor, Aquaculture 437, (2015): 327). Co-administration of the 2 mint extracts as an emulsion reduced the concentration required for each drug by 75% suggesting possible drug synergy action, at least in fish (Z. Roohi & M. R. Imanpoor, J. Aquac. Res. Development 5, (2014): 1). The rat pilot experiments described herein with L-carvone, methyl salicylate, and the 1:1 administration of both compounds did not show as obvious a difference in dose requirement, onset of action, or recovery time. These similar anesthetic effects reflect similar receptor drug actions and potency at NMDA receptors and Naᵥ1.2 channels.

A significant difference in the mechanism of action between mint extracts was observed at the GABA_{A} receptor. However, L-carvone caused dose-dependent inhibition, and methyl salicylate exhibited a biphasic effect on GABA_{A} receptor currents (FIGS. 5 and 6). This implies the presence of at least 2 separate salicylate ester allosteric binding sites on the GABA_{A} receptor. Negative allosteric modulation occurred at a high-affinity binding site with an IC50 of approximately 0.1 μM methyl salicylate. This site may be the same GABA_{A} binding site as for the high affinity anesthetic ligands propofol and propanidid, which share structural similarities to L-carvone and methyl salicylate (H. Tsuchiya, Molecules 22, (2017): 1369). At about 50,000 times higher concentration, methyl salicylate binds a second, noncompetitive positive allosteric site that, at a saturated aqueous phase concentration, produced a predicted GABA_{A} current potentiation to equal to half of the maximum observable enhancement possible with the agonist concentration used in this study. This binding site may be similar to the amphipathic, water-filled, allosterically active pockets predicted for low-affinity interactions with inhaled anesthetics and other simple hydrocarbons (R. J. Brosnan & T. L. Pham, BMC Pharmacol. Toxicol. 15, (2014): 62; R. J. Brosnan & T. L. Pham, BMC Pharmacol. Toxicol. 19, (2018): 57).

GABA_{A} receptor potentiation typically causes central nervous system depression; GABA_{A} receptor inhibition causes convulsions (E. I. Eger 2nd et al., Anesth. Analg. 88, (1999): 884). During anesthesia maintenance, L-carvone caused tonic muscle contractions suggestive of seizure activity at concentrations corresponding to near total inhibition of GABA_{A} receptor currents. Although methyl salicylate also inhibits these same currents at low concentrations, a rapid rise in plasma drug concentration to agonist levels during IV administration probably circumvented seizures in these rats. Additionally, inhibition of glutamate receptors and sodium channel currents decreases neuronal excitability and may have stopped seizure activity during L-carvone anesthesia and prevented visible seizures following high-dose bolus euthanasia in pilot rat experiments (E. J. Nestler, P. J. Kenny, S. J. Russo, & A. Shaefer, Molecular neuropharmacology: a foundation for clinical neuroscience. 4th ed. New York, NY: McGraw-Hill, (2020): 608).

Differences between mint extract effects on GABA_{A} receptors may also explain differences in drug potency. GABA_{A} receptor inhibition increases neuronal excitability and increases anesthetic median effective concentrations (Y. Zhang, C. Stabernack, J. Sonner, R. Dutton, & E. I. Eger 2nd, Anesth. Analg. 92, (2001): 1585). Presumably, this is because increased basal excitability must be overcome through more depressant effects at other anesthetic-sensitive receptor targets, such as from NMDA receptor inhibition (R. J. Brosnan, Vet. Anaesth. Analg. 38, (2011): 231). At the time general anesthesia was achieved and the aorta catheterized, plasma concentration of L-carvone was 3 times that for methyl salicylate and corresponded to GABA_{A} receptor inhibition for L-carvone but GABA_{A} receptor potentiation for methyl salicylate. GABA_{A} receptors would therefore contribute to methyl salicylate anesthesia but antagonize L-carvone anesthesia. These higher L-carvone plasma concentrations during anesthesia equate to 15-30% greater inhibition of NMDA receptors and Naᵥ1.2 channels than was present at the methyl salicylate plasma concentrations (FIGS. 7-10). Increased action at these and perhaps other receptor targets allows L-carvone to induce an anesthesia effect despite contrary activity at GABA_{A} receptors. Indeed, a similar phenomenon has been described for conventional anesthetics, such as the inhaled agents isoflurane and sevoflurane that exhibit low-affinity interactions with structurally diverse anesthetic-sensitive ion channels and receptors. Antagonism of spinal GABA_{A} receptors in rats increases the isoflurane EC50 for immobilization by up to 40% (Y. Zhang, C. Stabernack, J. Sonner, R. Dutton, & E. I. Eger 2nd, Anesth. Analg. 92, (2001): 1585). However, isoflurane requirement is not greater with further GABA_{A} receptor inhibition, presumably because increased isoflurane concentrations produce increased effects at other molecular targets—such as NMDA receptors (J. Yang & C. F. Zorumski, Ann. N. Y. Acad. Sci. 625, (1991): 287), voltage-gated sodium channels (M. Shiraishi & R. A. Harris, J. Pharmacol. Exp. Ther. 309, (2004): 987), 2-pore domain potassium channels (A. J. Patel et al., Nat. Neurosci. 2, (1999): 422), and glycine receptors (D. L. Downie, A. C. Hall, W. R. Lieb, & N. P. Franks, Br. J. Pharmacol. 118, (1996): 493)—that combined are sufficient to depress CNS function and cause immobility. Similarly, antagonism of spinal glycine receptors in rats increases the sevoflurane EC50, at which general anesthesia is mediated by greater inhibition of NMDA receptors produced by this greater sevoflurane concentration (R. J. Brosnan & R. Thiesen, BMC Anesthesiol. 12, (2012): 9).

Although the Examples provided herein focused on an anesthetic endpoint, these same receptor systems exert other important neurophysiologic effects. Sodium channel blockers and glutamate receptor antagonists act at sites within both the peripheral and central nervous system where they decrease pain (E. J. Nestler, P. J. Kenny, S. J. Russo, & A. Shaefer, Molecular neuropharmacology: a foundation for clinical neuroscience. 4th ed. New York, NY: McGraw-Hill, (2020): 608). In addition, both mint extracts can modulate other anesthetic-sensitive ion channels important for analgesia, such as the transient receptor potential cation channel TRPV1, achieving antinociceptive effects at subanesthetic drug concentrations.

Although the foregoing disclosure has been described in some detail by way of illustration and example for purpose of clarity of understanding, one of skill in the art will appreciate that certain changes and modifications within the spirit and scope of the disclosure may be practiced, e.g., within the scope of the appended claims. It should also be understood that aspects of the disclosure and portions of various recited embodiments and features can be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the disclosure. In addition, each reference provided herein is incorporated by reference in its entirety for all purposes to the same extent as if each reference was individually incorporated by reference.

What is claimed is:

1. A method of inducing anesthesia in a subject, the method comprising administering to the subject an amount of a compound selected from the group consisting of a terpenoid, a salicylate, and a combination thereof, wherein the amount is effective for inducing anesthesia in the subject.

2. The method of claim 1, wherein the compound is a Type II voltage-gated sodium channel antagonist.

3. The method of claim 1 or 2 wherein the compound is an N-methyl-D-aspartate receptor antagonist.

4. The method of any one of claim 1-3, wherein the compound is a γ-aminobutyric-acid-A receptor agonist.

5. The method of any one of claims 1-4, wherein the compound is a terpenoid.

6. The method of claim 5, wherein the terpenoid has only one isoprene unit.

7. The method of claim 5, wherein the terpenoid has only two isoprene units.

8. The method of claim 7, wherein the terpenoid is L-carvone.

9. The method of claim 5, wherein the terpenoid has only three isoprene units.

10. The method of any one of claims 1-3, wherein the compound is a salicylate.

11. The method of claim 10, wherein the salicylate is an ester of salicylic acid.

12. The method of claim 11, wherein the salicylate is selected from the group consisting of methyl salicylate, ethyl salicylate, and benzyl salicylate.

13. The method of any one of claims 1-12, wherein the administering is via injection.

14. The method of claim 13, wherein the injection is intravenous, intraperitoneal, intramuscular, or subcutaneous.

15. The method of any one of claims 1-14, wherein the subject is a mammal.

16. The method of any one of claims 1-15, wherein the anesthesia has an endpoint comprising analgesia, tranquilization, sedation, amnesia, a hypnotic state, a state of insensitivity to noxious stimulation, or a combination thereof.

17. The method of any one of claims 1-15, wherein the effective amount comprises a dosage less than or equal to 2 mg/kg.

18. The method of claim 17, wherein the anesthesia has an endpoint comprising analgesia.

19. The method of any one of claims 1-15, wherein the effective amount comprises a dosage of from 2 mg/kg to 4 mg/kg.

20. The method of claim 19, wherein the anesthesia is general anesthesia.

21. The method of any one of claims 1-15, wherein the effective amount comprises a dosage greater than or equal to 4 mg/kg.

22. The method of claim 21, wherein the anesthesia has an endpoint comprising euthanasia.

23. The method of any one of claims 15-22, wherein the mammal is a livestock animal used for meat, milk, or wool production.

24. The method of any one of claims 15-22, wherein the mammal is a dog, cat, rabbit, rodent, horse, or other companion animal.

25. The method of any one of claims 15-20, wherein the mammal is a human.

26. The method of any one of claims 1-25, wherein the compound is formulated in a composition comprising a delivery vehicle.

27. The method of claim 26, wherein the delivery vehicle comprises a lipid emulsion.

28. The method of claim 26 or 27, wherein the delivery vehicle comprises a cyclodextrin.

29. A composition for inducing anesthesia in a subject, the composition comprising:
an amount of a compound selected from the group consisting of a terpenoid, a salicylate, and a combination thereof, wherein the amount is effective for inducing anesthesia in the subject; and
delivery vehicle.

30. The composition of claim 29, wherein the compound is a Type II voltage-gated sodium channel antagonist.

31. The composition of claim 29 or 30 wherein the compound is a N-methyl-D-aspartate receptor antagonist.

32. The composition of any one of claims 29-31, wherein the compound is a γ-aminobutyric-acid-A receptor agonist.

33. The composition of any one of claims 29-32, wherein the compound is a terpenoid.

34. The composition of claim 33, wherein the terpenoid has only one isoprene unit.

35. The composition of claim 33, wherein the terpenoid has only two isoprene units.

36. The composition of claim 35, wherein the terpenoid is L-carvone.

37. The composition of claim 33, wherein the terpenoid has only three isoprene units.

38. The composition of any one of claims 29-31, wherein the compound is a salicylate.

39. The composition of claim 38, wherein the salicylate is an ester of salicylic acid.

40. The composition of claim 39, wherein the salicylate is selected from the group consisting of methyl salicylate, ethyl salicylate, and benzyl salicylate.

41. The composition of any one of claims 29-40, wherein the subject is a mammal.

42. The composition of any one of claims 29-41, wherein the anesthesia has an endpoint comprising analgesia, tranquilization, sedation, amnesia, a hypnotic state, a state of insensitivity to noxious stimulation, or a combination thereof.

43. The composition of any one of claims 29-41, wherein the effective amount comprises a dosage less than or equal to 2 mg/kg.

44. The composition of claim 43, wherein the anesthesia has an endpoint comprising analgesia.

45. The composition of any one of claims 29-41, wherein the effective amount comprises a dosage of from 2 mg/kg to 4 mg/kg.

46. The composition of claim 45, wherein the anesthesia is general anesthesia.

47. The composition of any one of claims 29-41, wherein the effective amount comprises a dosage greater than or equal to 4 mg/kg.

48. The composition of claim 47, wherein the anesthesia has an endpoint comprising euthanasia.

49. The composition of any one of claims 41-48, wherein the mammal is a livestock animal used for meat, milk, or wool production.

50. The composition of any one of claims 41-48, wherein the mammal is a dog, cat, rabbit, rodent, horse, or other companion animal.

51. The composition of any one of claims 41-46, wherein the mammal is a human.

52. The composition of any one of claims 29-51, wherein the delivery vehicle comprises a lipid emulsion.

53. The composition of any one of claims 29-52, wherein the delivery vehicle comprises a cyclodextrin.
